(19) United States
(12) Patent Application Publication
(10) Pub. No.: US 2007/0270887 A1
(43) Pub. Date: Nov. 22, 2007

(54) CIRCULAR NEEDLE PUSHER

(76) Inventor: Orhan Seyfi AKSAKAL, ANKARA (TR)

Correspondence Address: Oppedahl Patent Law Firm LLC, P.O. BOX 4850, FRISCO, CO, 80443-4850, US

(21) Appl. No.: 11/609,929

(22) Filed: Dec. 13, 2006

(30) Foreign Application Priority Data
May 16, 2006 (TR) 2006/02413

(51) Int. Cl. A61B 17/04

(52) U.S. Cl. 606/144

(57) ABSTRACT

The circular needle pusher has a body (1), handle (2), needle pushing trigger connecting piece (3), needle pushing trigger (4), spindle (5), needle capturing trigger (6), rotation point (7), lock mechanism (8), needle holder control arm (9), cap (10), cap rotation point (11), needle holder jaws (12), fixing arm (13), pusher arm (14), needle slot (15), pusher arm slot (16), slot rotation point (17), slot spindle (18), needle (19) and needle canal (20).

### TECHNICAL FIELD OF THE INVENTION AND THE BACKGROUND ART

The invention will be used for the laparoscopic atraumatic suture procedure. In the current state of the art, this procedure is carried out using the traditional needle holders.

### TECHNICAL PROBLEMS THE INVENTION AIMS TO SOLVE

Currently, single-camera imaging systems are used in laparoscopic surgery, and this leads to difficulties with the needle holder currently available during the steps like capturing the needle and bringing it to suitable position. Normally, first the needle is put inside (the abdominal cavity), then it is captured with the needle holders to carry out the suture. With the invention, the solution is provided for the steps of putting first the needle inside, capturing the needle, performing the suture, capturing after the suture of the meat and withdrawal of the same.

### DESCRIPTION OF THE DRAWINGS

FIG. 1 is a side view.

FIG. 2 is a side perspective view of the handle, needle pushing trigger and needle capturing trigger.

FIG. 3 is a side view of the lock mechanism.

FIG. 4 is a perspective view of the handle, needle capturing trigger and the lock mechanism.

FIG. 5 shows a pusher arm.

FIG. 6 shows a rear section of the pusher arm.

FIG. 7 shows a front section of the pusher arm.

FIG. 8 shows a needle holder control arm.

FIG. 9 shows the needle holder jaws in closed state.

FIG. 10 shows the needle holder jaws in open state.

FIG. 11 shows the front part of the body (without the needle being attached).

FIG. 12 shows the front part of the body (with the needle attached), needle tightened between the needle holder jaws.

FIG. 13 shows the front part of the body (with the needle attached), needle tightened between the needle holder jaws, the view from a different angle.

FIG. 14 shows the front part of the body (with the needle attached), needle slot in open state.

FIG. 15 shows the needle slot.

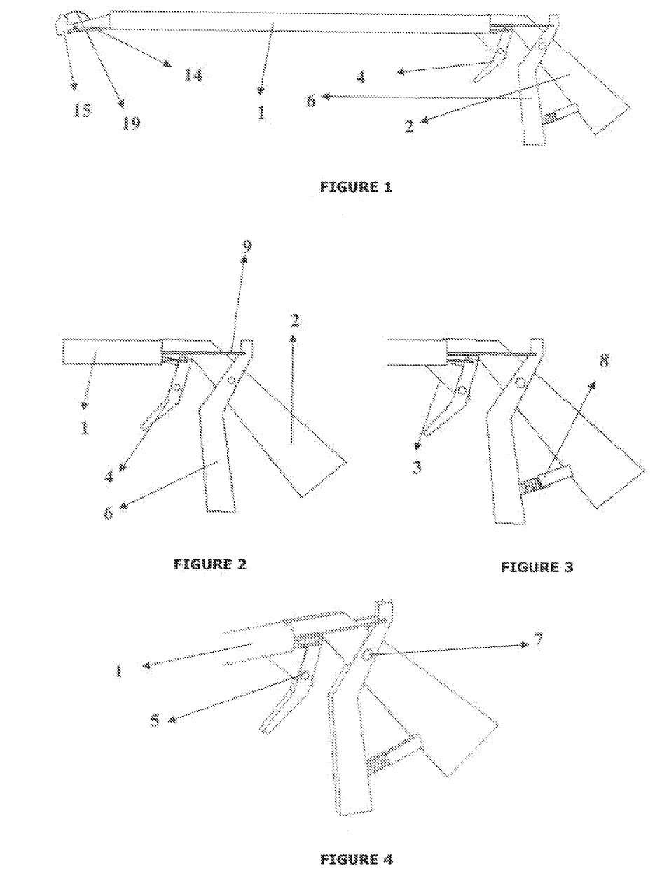

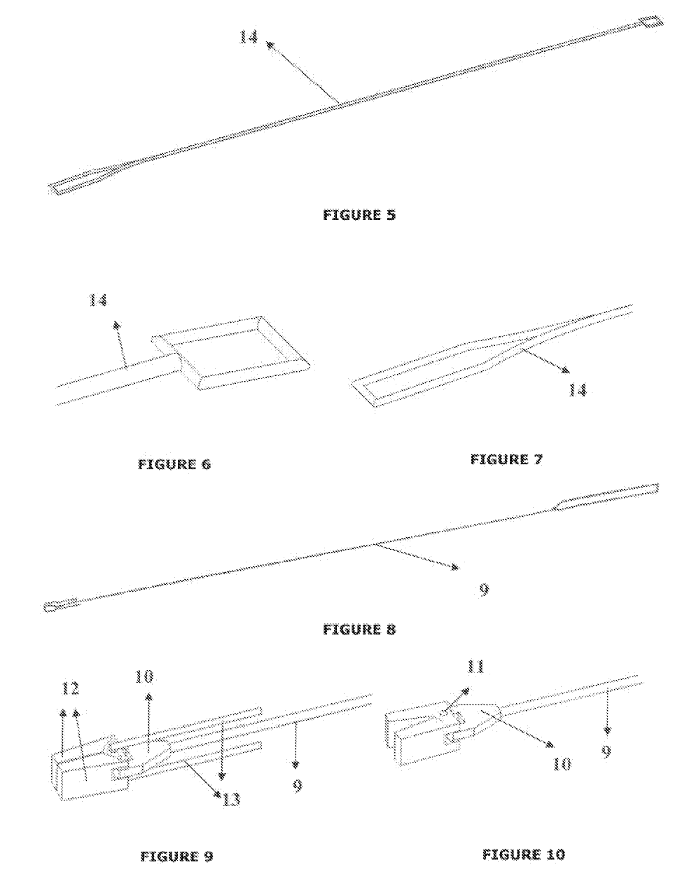

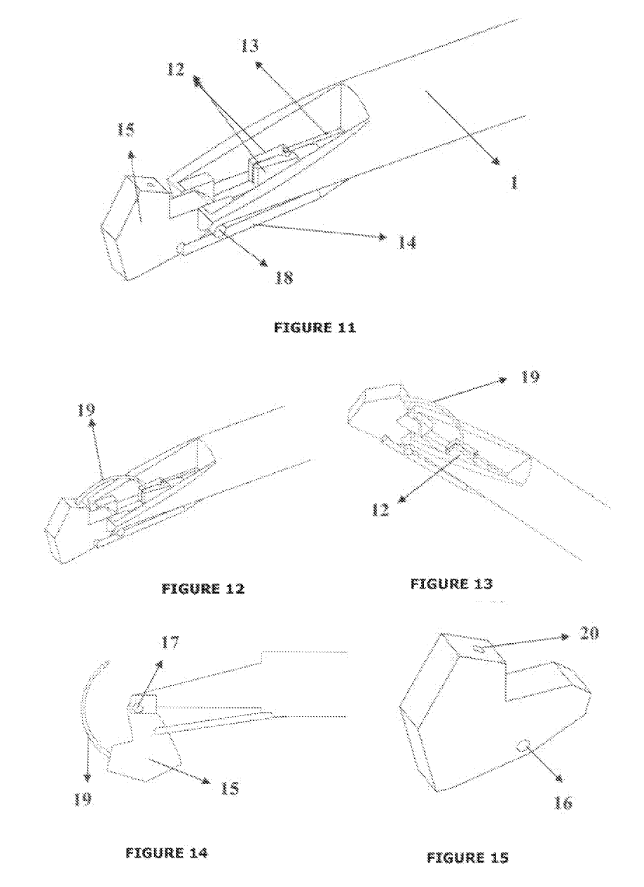

### DETAILED DESCRIPTION

For convenient reference a part number list is provided:

### PART NUMBERS

1. Body
2. Handle
3. Needle pushing trigger connecting piece
4. Needle pushing trigger
5. Spindle
6. Needle capturing trigger
7. Rotation point
8. Lock mechanism
9. Needle holder control arm
10. Cap
11. Cap rotation point
12. Needle holder jaws
13. Fixing arm
14. Pusher arm
15. Needle slot
16. Pusher arm slot
17. Slot rotation point
18. Slot spindle
19. Needle
20. Needle canal

The invention comprises the body (1), handle (2), needle pushing trigger connecting piece (3), needle pushing trigger (4), spindle (5), needle capturing trigger (6), rotation point (7), lock mechanism (8), needle holder control arm (9), cap (10), cap rotation point (11), needle holder jaws (12), fixing arm (13), pusher arm (14), needle slot (15), pusher arm slot (16), slot rotation point (17), slot spindle (18), needle (19) and needle canal (20).

The tubular body (1) with a diameter of 10 mm is connected on its rear part with the handle (2). On the lower part of the body, the needle pushing trigger connecting piece (3) is located such that it will be below the section where the handle is connected to the body. The needle pushing trigger (4) is connected to the needle pushing trigger connecting piece (3) by means of a spindle (5), which provides it with the possibility to rotate. The needle capturing trigger (6) is connected to the handle (2) through the rotation point (7). Lock mechanism (8) is located between the handle (2) and the needle capturing trigger (6).

At the end of the needle holder control arm (9), there are located the cap (10) and the needle holder jaws (12) connected to the cap by means of the cap rotation point (11). Backwards from the needle holder jaws (12), two fixing arms (13) protrude parallel to the needle holder control arm (9) that secures the needle holder jaws (12) to the body. The rear part of the needle holder control arm (9) is designed so that it will pass through the middle of the handle (2) and be connected with the needle capturing trigger (6). When the needle capturing trigger (6) is pulled backwards, the needle holder control arm moves forward and the cap (10) at its end imparts pressure on the needle holder jaws (12) from the back side, thus the jaws (12) come closer to one another from the cap rotation point (11) (the mouth of the jaws closes). When the needle capturing trigger (6) is pushed forward, the mouth of the jaws (12) opens.

The rear part of the pusher arm (14) is connected to the needle pushing trigger (4) and its front part is given a shape where the needle slot (15) can be mounted. The needle slot (15) is connected to the pusher arm (14) via the pusher arm slot (16), and also to the body (1) by means of the slot spindle (18) passing through slot rotation point (17). Also in the upper part of the needle slot (15), there is located the needle canal (20) suitable for the structure of the needle (19) and its insertion, where 1/3 of the needle may be inserted. When the needle pushing trigger (4) is pulled backwards, the pusher arm (4) moves forward and enables the needle slot (15) to rotate from the slot rotation point (17) and the needle slot (15) to close towards the body (1) with the needle (19) located between the needle holder jaws (12), owing to a vertical circular movement. When the needle pushing trigger (4) is pushed forwards, the needle slot (15) is released from the needle and returns, where the needle (19) remains between the needle holder jaws (12).

### THE APPLICATION OF THE INVENTION

The circular needle pusher will be used for the atraumatic suture procedures at sites with a depth up to 30 cm. By a single hold of the handle (2) having an ergonomic structure, the needle pushing trigger (4) is controlled with the index finger and the needle capturing trigger (6) is controlled with middle finger, third finger and the little finger (using all three fingers). At the initial state, the needle pushing trigger (4) is at the position of maximum pull, while the needle capturing trigger (6) is at the position of the maximum push. The body (1) of the circular needle pusher, where the needle (19) bears the yarn tied at its back and is inserted in the needle slot (15) being closed onto the body (1), is conveyed to the operation site inside the trocar with an inner width of 10 mm, the needle pushing trigger (4) is pushed up to its end to allow the needle slot (15) to rotate and open, and the needle tip is drawn near the tissue and brought to the location where the suture is to be carried out.

The needle pushing trigger (4) is pulled backwards, by which movement the needle slot (15) rotates forward, to enable the needle (19) with the yarn tied at its back to pass through the tissue and the needle tip to enter between the jaws (12).

The needle capturing trigger (6) is pulled backwards to enable the needle (19) to be tightened between the jaws (12). And the lock mechanism (8) is locked.

The needle pushing trigger (4) is pushed forward. Since the needle (19) is tightened between the jaws (12), the needle slot (15) is released from the needle (19) and returns in an empty state.

The body (1) is pulled outwards, with the yarn being passed through the tissue and connected with the needle. Because one end of the yarn is already outside, both ends of the yarn having passed through the tissue are now in the hands of the surgeon. Thus the suture procedure is achieved.

1. The circular needle pusher characterized in that it comprises the body (1), handle (2), needle pushing trigger connecting piece (3), needle pushing trigger (4), spindle (5), needle capturing trigger (6), rotation point (7), lock mechanism (8), needle holder control arm (9), cap (10), cap rotation point (11), needle holder jaws (12), fixing arm (13), pusher arm (14), needle slot (15), pusher arm slot (16), slot rotation point (17), slot spindle (18), needle (19) and needle canal (20).

2. The circular needle pusher according to claim 1 characterized in that it has the two-jaw (12) needle capturing system and that the needle holder jaws (12) may be controlled with the handle (2) via the capturing trigger (6).

3. The circular needle pusher according to claim 1 characterized in that it comprises the lock mechanism (8) between the handle (2) and the needle capturing trigger (6).

4. The circular needle pusher according to claim 1 characterized in that it comprises the needle slot (15) and the needle slot (15) is capable for performing a vertical circular movement to be closed onto the body (1) and said movement may be controlled with the handle (2) via the needle pushing trigger (4).

5. The circular needle pusher according to claim 1 characterized in that the body (1) may enter the operation site by passing together with the needle (19) inside a trocar with an inner width of 10 mm, with the needle slot (15) being closed towards the body.

6. The circular needle pusher according to claim 1 characterized in that by a single hold of the handle (2) having an ergonomic structure, the needle pushing trigger (4) may be controlled with the index finger and the needle capturing trigger (6) may be controlled with middle finger, third finger and the little finger (using all three fingers)
